(19) United States
(12) Patent Application Publication
(10) Pub. No.: US 2021/0094897 A1
(43) Pub. Date: Apr. 1, 2021

(54) IMPROVED PROCESS FOR PREPARING A CHLORINATED ALKENE BY CAUSTIC DEHYDROCHLORINATION OF A CHLORINATED ALKANE IN A JET LOOP REACTOR

(71) Applicant: Blue Cube IP LLC, Clayton, MO (US)

(72) Inventors: Max Tirtowidjojo, Clayton, MO (US); Michael Schluter, Clayton, MO (US); Marc Sell, Clayton, MO (US)

(73) Assignee: Blue Cube IP LLC, Clayton, MO (US)

(21) Appl. No.: 16/981,975

(22) PCT Filed: Apr. 3, 2019
(86) PCT No.: PCT/US2019/025337

§ 371 Date: Sep. 17, 2020

Related U.S. Application Data
(60) Provisional application No. 62/652,085, filed on Apr. 3, 2018.

(51) Int. Cl. C07C 17/25; C07C 17/20; B01J 19/24; B01J 19/26

(52) U.S. Cl. CPC C07C 17/25; B01J 19/26; B01J 19/246; C07C 17/202

(57) ABSTRACT

The present invention provides a process for preparing at least one chlorinated alkene from at least one chlorinated alkane, using an aqueous base in a jet loop reactor.

### FIELD OF THE INVENTION

The present disclosure generally relates to processes for preparing chlorinated alkenes.

### BACKGROUND OF THE INVENTION

Chlorinated alkenes are useful intermediates for many products including agricultural products, pharmaceuticals, cleaning solvents, gums, silicones, and refrigerants. One method for preparing chloroalkenes involves using a catalyst to dehydrochlorinate a chlorinated alkane. Common dehydrochlorination catalysts include Lewis acids, such as FeCl₃ or AlCl₃, which are not complexed with a ligand. The ligand can reduce the reaction rate and yield of the dehydrochlorination reaction. These catalysts have been useful for providing the chlorinated alkenes in good yields. Yet, additional purification protocols are necessary to remove the catalyst from the chlorinated alkene, which can inhibit later processes and increase costs.

Another method for producing a chlorinated alkene comprises contacting the chlorinated alkane with an aqueous base in a dehydrochlorination process. Generally, these processes are efficient, yet a co-solvent such as an alcohol provides miscibility of the organic and aqueous phases. Processes that use no co-solvent have been developed, but they are inefficient and require additional separation steps, which reduce the yield of the chlorinated alkene.

An improvement to the base dehydrochlorination process described above uses phase transfer catalyst to enhance the miscibility of the organic and aqueous phases and increase the kinetics of the dehydrochlorination reaction. But phase transfer catalysts are expensive and they are commonly purged to waste, which necessitates purchasing additional phase transfer catalyst. This increases the overall cost of the base dehydrochlorination process.

It would be desirable to develop a process for preparing a chlorinated alkene with increased reaction kinetics, low unit manufacturing cost, high purity, and enables efficient recycle strategies, including the recycling of unreacted chlorinated alkane and/or reagents.

### SUMMARY OF THE INVENTION

Provided herein are processes for dehydrochlorinating a chlorinated alkane in a jet loop reactor. To be clear, all dehydrochlorination reactions described herein are performed in a jet loop reactor. The process comprises treating at least one chlorinated alkane with an aqueous base, to form at least one chlorinated alkene. As is readily apparent to those skilled in the art, the chlorinated alkene product(s) depend on the chlorinated alkane or alkanes subjected to the dehydrochlorination reaction.

In another aspect, provided herein are processes of dehydrochlorinating 1,1,1,3-tetrachloropropane. The process comprises treating 1,1,1,3-tetrachloropropane with an aqueous base comprising 5-20 wt % NaOH, KOH or a combination thereof, to form 1,1,3-trichloropropene and 3,3,3-trichloropropene. The aqueous base may contain up to the saturated wt % of a halide salt, such as NaCl.

In a further aspect, provided herein are processes of dehydrochlorinating 1,1,1,2,3-pentachloropropane (240DB) with an aqueous base comprising 5-20 wt % NaOH, KOH or a combination thereof to form chlorinated alkenes comprising 1,1,2,3-tetrachloropropene, 2,3,3,3-tetrachloropropene, and combinations thereof. The aqueous base may contain up to the saturated wt % of a halide salt, such as NaCl.

In yet another aspect, provided herein are processes of dehydrochlorinating 1,1,1,3,3-pentachloropropane (240FA) with an aqueous base comprising 5-20 wt % NaOH, KOH or combinations thereof to form chlorinated alkenes comprising 1,1,3,3-tetrachloropropene, 1,3,3,3-tetrachloropropene, and combinations thereof. The aqueous base may contain up to the saturated wt % of a halide salt, such as NaCl.

Other features and iterations of the invention are described in more detail below.

### BRIEF DESCRIPTION OF THE FIGURES

FIG. 1 shows a schematic of jet loop reactor with a two-phase nozzle and a draft tube and an impinging plate below the draft tube.

### DETAILED DESCRIPTION OF THE INVENTION

Disclosed herein are dehydrochlorination processes that are conducted in a jet loop reactor. The processes dehydrochlorinate at least one chlorinated alkane to form at least one chlorinated alkene. Generally, the processes utilize an aqueous base.

### (I) Processes for Preparing Chlorinated Alkenes

The process for preparing at least one chlorinated alkene from at least one chlorinated alkane comprises treating at least one chlorinated alkane with an aqueous base to form at least one chlorinated alkene. Under process conditions described below, a high yield of the chlorinated alkene results.

### (a) Chlorinated Alkane

The at least one chlorinated alkane useful in this process may be a C₂-C₆ chlorinated alkane. The at least one chlorinated alkane may be selected from the group consisting of a dichlorinated propane, a trichlorinated propane, a tetrachlorinated propane, a pentachlorinated propane, a hexachlorinated propane, a tetrachlorinated ethane, trichlorinated ethane, dichlorinated ethane, and combinations thereof. Non-limiting examples of trichloropropanes, tetrachloropropanes, pentachloropropanes, and hexachloropropanes include 1,1-dichloropropane; 1,2-dichloropropane; 1,3-dichloropropane; 1,1,1-trichloropropane; 1,1,22-trichloropropane; 1,2,2-trichloropropane; 1,2,3-trichloropropane; 1,1,1,2-tetrachloropropane; 1,1,2,2-tetrachloropropane; 1,1,1,3-tetrachloropropane; 1,1,2,3-tetrachloropropane; 1,1,3,3-tetrachloropropane; 1,1,1,2,3-pentachloropropane; 1,1,2,3,3-pentachloropropane; 1,1,2,2,3-pentachloropropane; 1,1,1,3,3-pentachloropropane; 1,1,1,3,3,3-hexachloropropane; 1,1,2,2,3,3-hexachloropropane; or combinations thereof.

In a preferred embodiment, the at least one chlorinated alkane comprises a chlorinated propane or a chlorinated ethane. In one embodiment, the at least one chlorinated alkane comprises 1,1,1,3-tetrachloropropane (250FB); 1,1,1,2,3-pentachloropropane (240DB); or 1,1,1,3,3-pentachloropropane (240FA).

One method for preparing these chlorinated alkanes is through the telomerization process. In this process, carbon tetrachloride (Tet), an alkene or chlorinated alkene, a catalyst system comprising metallic iron, ferric chloride, and/or ferrous chloride, and a trialkylphosphate and/or a trialkylphosphite are contacted to produce the chlorinated alkanes. As an illustrative example, using ethylene as the monomer in the above-described telomerization process yields tetrachloropropanes. Using vinyl chloride as the monomer, pentachloropropanes result. The skilled artisan readily knows other methods for preparing chlorinated alkanes.

The chlorinated alkane may be crude, unpurified product from the telomerization reaction, partially purified, or fully purified by means known to the skilled artisan. One common method of purification the chlorinated alkane is distillation. Non-limiting examples of distillations may be a simple distillation, flash distillation, a fractional distillation, a steam distillation, or a vacuum distillation. If necessary or desired, multiple distillations may be used to achieve a desired purity level.

Generally, the chlorinated alkane useful in the process may have a purity greater than 10 wt %. In various embodiments, the purity of the chlorinated alkane may have a purity greater than 10 wt %, greater than 30 wt %, greater than 50 wt %, greater than 75 wt %, greater than 90 wt %, greater than 95 wt %, or greater than 99 wt %. As a general rule, purer chlorinated alkanes are preferred.

### (b) Aqueous Base

The dehydrochlorination process uses an aqueous base. In an embodiment, the aqueous base may be an inorganic base. The aqueous base may further contain an inorganic halide salt, such as a chloride salt, and more preferably NaCl. In an embodiment, the aqueous phase comprising an aqueous base may be produced by the chloroalkali process.

The inorganic base may be an alkali or alkali earth metal base. Non-limiting examples of these alkali or alkali earth bases may be lithium hydroxide (LiOH), sodium hydroxide (NaOH), potassium hydroxide (KOH), barium hydroxide (Ba(OH)₂), calcium hydroxide (Ca(OH)₂), sodium carbonate (Na₂CO₃), potassium carbonate (K₂CO₃), sodium bicarbonate (NaHCO₃), potassium bicarbonate (KHCO₃), or combinations thereof. In a preferred embodiment, the alkali or alkali earth metal base may be sodium hydroxide, potassium hydroxide, or combinations thereof. Still more preferably, the base comprises sodium hydroxide.

The halide salt may be any alkali or alkali earth metal halide salt. Non-limiting examples of these alkali or alkali earth metal salt halide salts may be selected from a group consisting of lithium chloride, sodium chloride, potassium chloride, barium chloride, calcium chloride, or combinations thereof. In one embodiment, the salt may comprise sodium chloride (NaCl), potassium chloride (KCl), calcium chloride (CaCl₂), and combinations thereof. In some embodiments, the source of aqueous base is one or more cell effluents selected from the group consisting of a diaphragm cell, a membrane cell, and combinations thereof. In a preferred embodiment, the chloride salt is sodium chloride. In another embodiment, an aqueous base comprises a mixture of NaOH and at least one chloride salt produced from the chloralkali process through the electrolysis of sodium chloride in a diaphragm cell.

Generally, the concentration of the aqueous base may range from 5 wt % to about 50 wt %. In various embodiments, the concentration of the aqueous base may range from 5 wt % to about 50 wt %, from 7 wt % to about 40 wt %, from 9 wt % to about 30 wt %, or from 10 wt % to about 20 wt %. In a preferred embodiment, the concentration of the aqueous base may range from 5 wt % to about 10 wt %.

In general, the mole ratio of the base(s) to the chlorinated alkane may range from 0.1:1.0 to about 2.0:1.0. In various embodiments, the mole ratio of the base(s) to the chlorinated alkane may range from 0.1:1.0 to about 2.0:1.0, from 0.5:1.0 to about 1.5:1.0, or from 0.9:1.0 to about 1.1:1.0. In a preferred embodiment, the mole ratio of the aqueous base to the chlorinated alkane may be about 1.0:1.0.

In general, the concentration of the halide salt may be up to a saturated wt %. In various embodiments, the concentration of the halide salt may be greater than 0.01 wt %, greater than 1 wt %, greater than 10 wt %, greater than 20 wt %, or at the saturation limit for the suitable halide salt.

### (c) Chlorinated Alkenes

The process produces at least one chlorinated alkene. In various embodiments, the one or more chlorinated alkenes may comprise between 2 to 6 carbon atoms and may be linear, branched or cyclic. In some embodiments, the chlorinated alkene is a chlorinated propene. Non-limiting examples of chlorinated propenes include monochlorinated propenes, dichlorinated propenes, trichlorinated propenes, tetrachlorinated propenes, pentachlorinated propenes, or combinations thereof. Non-limiting examples of linear chlorinated alkenes include vinyl chloride, allyl chloride, 2-chloropropene, 3-chloropropene, 1,3-dichloropropene, 2,3-dichloropropene, 3,3-dichloropropene, 1,2,3-trichloropropene, 1,1,3-trichloropropene, 3,3,3-trichloropropene, 1,1,2,3-tetrachloropropene, 2-chloro-1-butene, 3-chloro-1-butene, 2-chloro-2-butene, 1,4-dichloro-2-butene, 3,4-dichloro-1-butene, 1,3-dichloro-2-butene, 2,3,4-trichloro-1-butene, 1,2,3,4-tetrachloro-2-butene, 1,1,2,4-tetrachloro-1-butene, 2,3-dichloro-1,3-butadiene, 1-chloro-3-methyl-2-butene, 3-chloro-3-methyl-butene, 5-chloro-1-pentene, 4-chloro-1-pentene, 3-chloro-1-pentene, 3-chloro-2-pentene, 1,2-dichloro-1-pentene, 1,1,5-trichloro-1-pentene, 6-chloro-1-hexene, 1,2-dichloro-1-hexene, and combinations thereof. Non-limiting examples of cyclic chlorinated alkenes include 1-chlorocyclopentene, 2-chlorocyclopentene, 3-chlorocyclopentene, 1,2-dichlorocyclopentene, 4,4-dichlorocyclopentene, 3,4-dichlorocyclopentene, 1-chloro-1,3-cyclopentadiene, 2-chloro-1,3-cyclopentadiene, 5-chloro-1,3-cyclopentadiene, 1,2-dichloro-1,3-cyclopentadiene, 1,3-dichloro-1,3-cyclopentadiene, 1,4-dichloro-1,3-cyclopentadiene, 5,5-dichloro-1,3-cyclopentadiene, 1,2,3-trichloro-1,3-cyclopentadiene, 1,2,3,4-tetrachloro-1,3-cyclopentadiene, 1-chloro-1,3-cyclohexadiene, and 3-chloro-1,4-cyclohexyldiene. As will be apparent to the person skilled in the art, the starting chlorinated alkane dictates which chlorinated alkene or alkenes will be formed.

In one embodiment, the chlorinated alkene comprises 1,1,3-trichloropropene, 3,3,3-trichloropropene, or combinations thereof. In another embodiment, the chlorinated alkene comprises 1,1,2,3-tetrachloropropene; 2,3,3,3-tetrachloropropene, or combinations thereof. In still other embodiments, the chlorinated alkene comprise 1,1,3,3-tetrachloropropene; 1,3,3,3-tetrachloropropene, or combinations thereof.

### (d) Reaction Conditions

In general, the dehydrochlorination reaction is a liquid phase reaction. The reaction commences by contacting the at least one chlorinated alkane (either purified, partially purified, or unpurified) and an aqueous base in a jet loop reactor. The components of the process may be added in any order. All the components of the process are typically mixed at a temperature enabling high yield of the chlorinated alkene product. Preferably, minimal byproducts are formed.

If desired, a phase transfer catalyst may be included in the reaction mixture. Non-limiting examples of phase transfer catalysts may be quaternary ammonium salts, phosphonium salts, pyridinium salts, or combinations thereof. In some embodiments, the phase transfer catalyst may be a quaternary ammonium salt. Non-limiting examples of suitable salts may be chloride, bromide, iodide, or acetate. Non-limiting examples quaternary ammonium salts may be trioctylmethylammonium chloride (Aliquat 336), trioctylmethylammonium bromide, dioctyldimethylammonium chloride, dioctyldimethylammonium bromide, Arquad 2HT-75, benzyldimethyldecylammonium chloride, benzyldimethyldecylammonium bromide, benzyldimethyldecylammonium iodide, benzyldimethyltetradecylammonium chloride, dimethyldioctadecylammonium chloride, dodecyltrimethylammonium chloride, tetrabutylammonium chloride, tetrabutylammonium bromide, tetrabutylammonium iodide, tetrabutylammonium acetate, tetrahexylammonium chloride, tetraoctylammonium chloride, tridodecylmethylammonium chloride, tetraethylammonium chloride, tetraethylammonium bromide, tetraethylammonium iodide, or combinations thereof. Non-limiting examples of phosphonium salts may be tetrabutylphosphonium bromide, dimethyldiphenyl phosphonium iodide, tetramethylphosphonium chloride, tetraphenylphosphonium bromide, trihexyltetradecylphosphonium chloride, or combinations thereof. Non-limiting examples of pyridinium salts may be cetylpyridinium chloride, hexadecylpyridinium bromide, hexadecylpyridinium chloride monohydrate, or combinations thereof. In a preferred embodiment, the phase transfer catalyst may be trioctylmethylammonium chloride (Aliquat 336).

Generally, the amount of the phase transfer catalyst may range from 0.05 wt % to about 5.0 wt % based on the weight of the components. In various embodiments, the amount of the phase transfer catalyst may range from 0.05 wt % to less than 5 wt %, from 0.1 wt % to 2.5 wt %, from 0.3 wt % to about 1 wt %, or from 0.4 wt % to about 0.7 wt %.

As appreciated by the skilled artisan, many methods adequately stir a process. In one embodiment, jet mixing is used. Typically, jet mixing can be accomplished using one or more nozzles, one or more eductors, or one or more jet loop reactors. Non-limiting examples of jet loop reactors include compact, impingement jet, and jet zone loop reactors. Jet loop reactors per se and their structure are known.

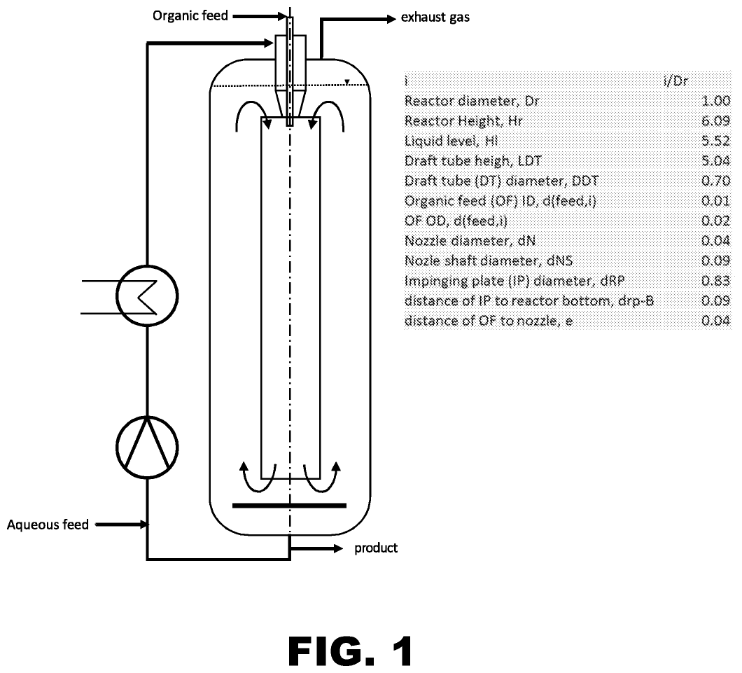

Referring to FIG. 1, a jet loop reactor generally contains one or more nozzles 2, one or more draft tubes 3, having a top, a bottom and a diameter, optionally one or more feed lines 4,6, optionally one or more product outlets 9, optionally one or more impingement plates 10, optionally one or more external circuits 5, and optionally further structures, for example coolers, in particular jacket coolers. The external circuit 5 connects the effluent stream to the input for the reactor, so unreacted starting material or byproducts can be brought back into the reactor for further treatment. An external circuit 5 comprises, for example, one or more pumps 7, one or more metering lines 6 and optionally one or more heat exchangers 8.

In one embodiment, the ratio of the reactor height to the reactor diameter is at least 5 and the ratio of the draft tube height to the reactor diameter is at least 4. Further, the ratio of the draft tube inner diameter to the reactor inner diameter is at least 0.6.

In another embodiment, the nozzle has an inner diameter and the ratio of nozzle inner diameter to the diameter of the reactor is at least 0.02.

In still another embodiment, the impinging plate has a diameter and the ratio of the impinging plate diameter to inner diameter of the reactor is less than 0.9.

In another embodiment, the jet loop reactor contains one or more nozzles, one or more draft tubes, optionally one or more feed lines, optionally one or more product outlets, optionally one or more impingement plates, optionally one or more external circuits and optionally further structures.

Referring again to FIG. 1, the nozzle 2 may be located entirely or partly within the jet loop reactor 1. Nozzle 2, draft tube 3 and optionally the impingement plate 10 may be arranged linearly, preferably in a vertical alignment, relative to one another. Owing to this arrangement, the reaction mixture is, after introduction via the nozzle 2, conveyed through the draft tube 3 and deflected in the region of the reactor bottom or the impingement plate 10 and the reaction mixture flows further through the region between draft tube 3 and reactor wall, so a flow 31, 32 is induced within the jet loop reactor 1 around the draft tube 3. As a result, the reaction mixture is circulated in the reactor 1.

In one embodiment, the nozzle outlet is placed within one diameter of the draft tube above or below the top of the draft tube. In another embodiment, the nozzle outlet is at the top of the draft tube. In these embodiments, the flow from the nozzle is directed downward into the top of the draft tube.

If the jet loop reactor 1 has a plurality of nozzles 2, these are preferably arranged next to one another, in particular parallel or horizontally. If a plurality of draft tubes 3 is present, these are also preferably arranged next to one another, in particular parallel or horizontally.

One or more external circuits 5 may be connected to the jet loop reactor 1 when part of the reaction mixture is removed from the jet loop reactor 1 and recirculated to the jet loop reactor 1. The reaction mixture can be removed at any point in the jet loop reactor 1. The reaction mixture is preferably removed at the bottom of the jet loop reactor 1, in particular below the impingement plate 10. From the external circuit 5, the reaction mixture is preferably recirculated into the jet loop reactor 1 through the nozzle 2. In this way, homogeneous mixing of the reaction mixture can be achieved. Further starting materials can be introduced via an organic feed 4 or aqueous feed 6. The organic feed can also added to the recycle line or circuit 5 before the pump as the aqueous feed. Optionally, the organic feed can also be introduced into the external circuit 5 as well. The temperature of the reaction mixture present in the external circuit 5 can be controlled with one or more heat exchangers 8. The transport of the reaction mixture through the external circuit 5 can be effected with one or more pumps 7.

The components of the jet loop reactor 1 may be arranged to effect desired reaction conditions. In FIG. 1, the nozzle 2 outlet is placed slightly above or below, but preferably at the entrance of the draft tube 3.

Generally, the process employs a driving jet velocity of at least 0.1 m/s. For example, the driving jet velocity may be greater than 1 m/s, and preferably greater than 5 m/s, and more preferably greater than 10 m/s.

The jet loop reactor can be operated by batch, semibatch or continuous processes, optionally with recirculation. In particular, the continuous process can also be operated without recirculation. In exemplary embodiments, the process is a continuous process.

In continuous operation without recirculation, starting materials are introduced via the organic feed 4 and aqueous feed 6 during reaction. The reaction mixture is discharged via the product outlet 9 and the product can then be isolated from this discharged reaction mixture. In continuous operation, the inflowing mass flows should correspond to the outflowing mass flows. In continuous operation without recirculation, preference is thus given to no external circuit 5 being installed.

In semibatch processes or in batch processes, one or more external circuits 5 may be installed on the jet loop reactor 1. With semibatch processes, starting materials are introduced via the organic feed 4 and aqueous feed 6, but no reaction mixture is discharged via the product outlet 9. With batch processes, no starting material is introduced via the organic feed 4 and aqueous feed 6, during reaction and no reaction mixture is discharged via the product outlet 9.

The charging of the jet loop reactor 1 with starting materials or introducing starting materials into the jet loop reactor 1 may be effected via the nozzle 2. As nozzles 2, preference is given to multifluid nozzles, in particular two-fluid nozzles. Two-fluid nozzles have two inlets via which in one case a starting material and in the other case the reaction mixture of an external circuit 5 are preferably conveyed and an outlet through which the reaction mixture is introduced into the jet loop reactor 1.

In a preferred embodiment, the jet loop reactor 1 is built into a cascade comprising further reactors. A cascade contains at least two reactors connected in series. The reaction mixture can be removed from the first reactor at any point and fed into the second reactor at any point. With jet loop reactors, the reaction mixture is preferably removed from the product outlet 9 of the first reactor and conveyed into the second reactor. When the second reactor is a jet loop reactor, the reaction mixture is preferably introduced into the jet loop reactor the organic feed 4.

Preferred cascades contain two or more jet loop reactors; or one or more jet loop reactors and one or more jet zone loop reactors; or one or more jet loop reactors and one or more airlift loop reactors; or one or more jet loop reactors and one or more stirred vessels. In the cascades, each reactor can be installed in one or more external circuits 5.

Generally, the mass ratio of the liquid recycle mass flow to the liquid fresh flow is greater than or equal to 1. In various embodiments, the mass ratio of the liquid recycle mass flow to the liquid fresh flow is greater than or equal to 1, greater than 15, greater than 30, greater than 50, or greater than 100. In a preferred embodiment, the mass ratio of the liquid recycle mass flow to the liquid fresh flow is greater than 30.

The temperature of the process can and will vary depending on purity of the at least one chlorinated alkane, the base, and the concentration of the base. Generally, the temperature of the process may be generally from 20° C. to about 120° C. or about 40° C. to about 120° C. In various embodiments, the temperature of the process may be generally from 20° C. to about 120° C., from 40° C. to about 80° C., or from 50° C. to 70° C.

Temperature regulation, i.e. heating or cooling, can be effected with one or more heat exchangers 8 in the external circuit 5 or be attached directly on the jet loop reactor. For this purpose, conventional heat exchangers may be used, for example jacket coolers, jacket heaters, shell-and-tube heat exchangers, or plate heat exchangers.

In general, the pressure of the process may range from about 0 psig (101 Pascal) to about 1000 psig (6894757 Pascal). In various embodiments, the pressure of the process may range from 0 psig to about 1000 psig, from 10 psig to about 900 psig, from 20 psig to about 100 psig, or from 40 psig to about 60 psig. In a preferred embodiment, the pressure of the process may be about atmospheric pressure and the process may be conducted under an inert atmosphere such as nitrogen, argon, or helium.

Generally, the reaction may proceed for a sufficient period until the reaction is complete, as determined by any method known to the skilled artisan, such as chromatography (e.g., GC). The duration of the reaction may range from about 5 minutes to about 12 hours. In some embodiments, the duration of the reaction may range from about 5 minutes to about 10 hours, from about 30 minutes to about 9 hours, from about 1 hours to about 8 hours, or from about 4 hours to about 7 hours.

The selectivity to the desired chlorinated alkene can and will vary depending on the reaction conditions, base, and the purity level of the chlorinated alkane used. Generally, the selectivity to the chlorinated alkene may be greater than 70%. In various embodiments, the selectivity to the desired chlorinated alkene may be greater than 70%, greater than 80%, greater than 90%, or greater than 95%. In preferred embodiments, the selectivity to the desired chlorinated alkenes may range from 95% to 99%.

The chlorinated alkane fed to the above described process may be converted to the chlorinated alkene isomers in at least 30% conversion. In various embodiments, the conversion of chlorinated alkane to the chlorinated alkene isomers may be at least 50%, at least 60%, at least 75%, at least 85%, at least 95%, and at least 99%.

### (II) Separating Chlorinated Alkene Products and Recycling Product Streams

The next step in the process comprises separating purified chlorinated alkenes from the contents of the reactor, which comprise the chlorinated alkene, halide salt, water, lighter byproducts, heavier byproducts, and unreacted chlorinated alkane starting material. (Depending on the purity of the chlorinated alkane used, further components may be, for example, a trialkylphosphate, a trialkylphosphite, and/or iron hydroxide.)

The separation process commences by transferring at least a portion of the reactor contents to a separator or multiple separators. As appreciated by the skilled artisan, many separation techniques may be useful. Non-limiting examples of separation techniques include decantation, settling, filtration, separation, centrifugation, thin film evaporation, simple distillation, vacuum distillation, fractional distillation, or a combination thereof. The distillations may comprise at least one theoretical plate. Depending on the quality and purity of the chlorinated alkane, various separation processes may be employed in various orders.

In one embodiment, the reactor contents are transferred to a separation device, where the aqueous phase (comprising the salt and optionally iron hydroxide) and the organic phase (comprising the chlorinated alkene, unreacted chlorinated alkane, lighter byproducts, heavier byproducts, and optionally trialkylphosphate or trialkylphosphite) are separated. In the separation device, the aqueous phase can be withdrawn from near or the top of the vessel and the organic phase can be withdrawn from near the bottom of the vessel. In a further embodiment, the aqueous layer is decanted.

The organic phase is then transferred to a second separator. In an embodiment, the second separator may use at least one simple distillation, at least one vacuum distillation, at least one fractional distillation, or combinations thereof. The distillations may comprise at least one theoretical plate. After leaving the second separator, all or >97% of the remaining dissolved water is removed.

As appreciated by the skilled artisan, separating the purified chlorinated alkene from the organic phase would produce at least two product streams. In various embodiments, separating the purified chlorinated alkene may produce three, four, or more product streams depending on the separation device(s) used. When the organic phase is distilled to produce only two product effluent streams, i.e., product effluent streams (a) and (b), product effluent stream (a) comprises the chlorinated alkene and optionally, unreacted chlorinated alkane, while product effluent stream (b) comprises the unreacted chlorinated alkane and the heavy by-products. Stream (b) may further contain chlorinated alkene.

Generally, product effluent stream (a) may be further purified and thereby produce two additional product effluent streams (c) and (d) wherein product effluent stream (c) obtained as an overhead stream comprises the chlorinated alkene and product effluent stream (d), obtained as the bottom stream, comprising the unreacted chlorinated alkane. Product effluent stream (b) may also be further purified and thereby produce two additional product effluent streams (e) and (f) wherein product effluent stream (e) comprises the unreacted chlorinated alkane and product effluent stream (f) comprises heavy by-products.

To improve the efficiency of the process, various product effluent streams may be recycled back into the process. In various embodiments, at least a portion of the product effluent stream (b), product effluent stream (d), and/or product effluent stream (e) may be recycled to the jet loop reactor.

In another embodiment, at least a portion of product effluent stream (b), product effluent stream (d), and/or product effluent stream (e) may be mixed with fresh feed (comprising non-recycled chlorinated alkane and/or aqueous base) before recycling into the reactor in batch mode or continuous mode. In various embodiments, the recycle streams and fresh feed streams may be introduced into the reactor separately or mixed before entering the process. Introducing these fresh feeds into the reactor or mixing the recycle streams with fresh feeds increases the efficiency of the process, reduces the overall cost, maintains the kinetics, increase the through-put, and reduces the byproducts produced by the process. The amounts of the product effluent streams recycled to the reactor or fresh liquid feeds added to the reactor may be the same or different. One way to measure the amount of product effluent streams and/or fresh liquid feeds being added to the reactor is to identify the mass flow of the materials. The product effluent stream being recycled to the reactor has a product effluent stream mass flow, while the fresh liquid feeds being added to the reactor has a fresh liquid feed mass flow. Mass flows may be measured using methods known in the art.

Generally, the mass of the product effluent stream mass flow being recycled to the fresh liquid feed mass flow is adjusted to not only maintain the conversion of the process but also maintain the kinetics of the process. In one embodiment the ratio of the product effluent stream mass flow to the fresh liquid feed mass flow is about 1.

Product effluent stream (a) from the separator may have a yield of at least about 10%. In various embodiments, the first product stream comprising chlorinated alkene produced in the process may have a yield of at least about 20%, at least about 50%, at least about 70%, at least about 75%, at least about 80%, at least about 85%, at least about 90%, at least about 95%, or at least about 99%.

### (III) Preferred Embodiment: Process for Preparing of 1,1,3-Trichloropropene, 3,3,3-Trichloropropene, or Combinations Thereof

(a) Process for Preparing 1,1,3-trichloropropane, 3,3,3-trichloropropane, or Combinations Thereof

Another aspect of the present disclosure encompasses a process for preparing 1,1,3-trichloropropene, 3,3,3-trichloropropene, or combinations thereof. The process commences by preparing and reacting a mixture comprising 1,1,1,3-tetrachloropropane and an aqueous base in a jet loop reactor in the presence of a phase transfer agent catalyst (PTA). In one embodiment, the PTA comprises a quaternary ammonium salt such as Aliquat 336. The aqueous base is described above in Section (I)(b). In a preferred embodiment, the aqueous base comprises 5 to 10 wt % NaOH, KOH, or combinations thereof and up to saturated wt % of NaCl.

### (b) Reaction Conditions

The reaction conditions are described above in Section (I)(d).

(c) Output from the Process to Prepare 1,1,3-trichloropropene, 3,3,3-trichloropropene, or Combinations Thereof.

The 1,1,1,3-tetrachloropropane fed to the above described process may be converted to 1,1,3-trichloropropene, 3,3,3-trichloropropene, or combinations thereof in at least 30% conversion. In various embodiments, the conversion of 1,1,1,3-tetrachloropropane to 1,1,3-trichloropropene, 3,3,3-trichloropropene or combinations thereof may be at least 50%, at least 60%, at least 75%, at least 85%, at least 95%, and at least 99%.

The selectivity to 1,1,3-trichloropropene, 3,3,3-trichloropropene or combinations thereof can and will vary depending on the reaction conditions, base, the purity level of the 1,1,1,3-tetrachloropropane used, and the 1,1,3-trichloropropene; 3,3,3-trichloropropene, or combinations thereof produced. Generally, the selectivity to 1,1,3-trichloropropene, 3,3,3-trichloropropene, or combinations thereof may be greater than 70%. In various embodiments, the selectivity to the 1,1,3-trichloropropene, 3,3,3-trichloropropene, or combinations thereof may be greater than 70%, greater than 80%, greater than 90%, or greater than 95%. In preferred embodiments, the selectivity to the 1,1,3-trichloropropene, 3,3,3-trichloropropene or combinations thereof may range from 95% to 99%.

(d) Separating the 1,1,3-trichloropropene, 3,3,3-trichloropropene, or Combinations Thereof and Recycling Product Streams.

The process for separating the 1,1,3-trichloropropene, 3,3,3-trichloropropene, or combinations thereof from the reactor contents is described above in Section (II). Specific recycle streams useful in improving the efficiency of the process are described above in Section (II).

The first product effluent stream (a) from the separator comprises the 1,1,3-trichloropropene; 3,3,3-trichloropropene, or combinations thereof, in a yield of at least about 10%. In various embodiments, the yield is at least about 20%, at least about 50%, at least about 70%, at least about 75%, at least about 80%, at least about 85%, at least about 90%, at least about 95%, or at least about 99%.

### (IV) Preferred Embodiment: Process for Preparing of 1,1,2,3-Tetrachloropropene, 2,3,3,3-Tetrachloropropene, or Combinations Thereof

(a) Process for Preparing 1,1,2,3-tetrachloropropene, 2,3,3,3-tetrachloropropene, or combinations thereof.

Another aspect of the present disclosure encompasses a process for preparing 1,1,2,3-tetrachloropropene, 2,3,3,3-tetrachloropropene, or combinations thereof. The process commences by preparing and reacting a mixture comprising 1,1,1,2,3-pentachloropropane (240DB) and an aqueous base in a jet loop reactor in the presence of a phase transfer agent catalyst (PTA). In one embodiment, the PTA comprises a quaternary ammonium salt such as Aliquat 336. The aqueous base is described above in Section (I)(b). In a preferred embodiment, the aqueous base comprises 5 to 10 wt % NaOH, KOH, or combinations thereof and up to saturated wt % of NaCl.

### (b) Reaction Conditions

The reaction conditions are described above in Section (I)(d).

(c) Output from the Process to Prepare 1,1,2,3-tetrachloropropene, 2,3,3,3-tetrachloropropene, or Combinations Thereof.

The 1,1,1,2,3-pentachloropropane fed to the above described process may be converted to 1,1,2,3-tetrachloropropene, 2,3,3,3-tetrachloropropene, or combinations thereof in at least 50% conversion. In various embodiments, the conversion of 1,1,1,2,3-pentachloropropane to 1,1,2,3-tetrachloropropene, 2,3,3,3-tetrachloropropene, or combinations thereof may be at least 50%, at least 60%, at least 75%, at least 85%, at least 95%, and at least 99%.

The selectivity to 1,1,2,3-tetrachloropropene, 2,3,3,3-tetrachloropropene, or combinations thereof can and will vary depending on the reaction conditions, base, the purity level of the 1,1,1,2,3-pentachloropropane, and the 1,1,2,3-tetrachloropropene, 2,3,3,3-tetrachloropropene, or combinations thereof produced. Generally, the selectivity to 1,1,2,3-tetrachloropropene, 2,3,3,3-tetrachloropropene, or combinations thereof may be greater than 70%. In various embodiments, the selectivity to the 1,1,2,3-tetrachloropropene, 2,3,3,3-tetrachloropropene, or combinations thereof may be greater than 70%, greater than 80%, greater than 90%, or greater than 95%. In preferred embodiments, the selectivity to the 1,1,2,3-tetrachloropropene, 2,3,3,3-tetrachloropropene, or combinations thereof may range from 95% to 99%.

(d) Separation of the 1,1,2,3-tetrachloropropene, 2,3,3,3-tetrachloropropene, or Combinations Thereof and Recycling Product Streams.

The process for separating the 1,1,2,3-tetrachloropropene, 2,3,3,3-tetrachloropropene, or combinations thereof from the reactor contents is described above in Section (II). Specific recycle streams useful in improving the efficiency of the process are described above in Section (II).

The first product effluent stream (a) from the separator comprises the 1,1,2,3-tetrachloropropene, 2,3,3,3-tetrachloropropene, or combinations thereof, in a yield of at least about 10%. In various embodiments, the yield is at least about 20%, at least about 50%, at least about 70%, at least about 75%, at least about 80%, at least about 85%, at least about 90%, at least about 95%, or at least about 99%.

### (V) Preferred Embodiment: Process for Preparing of 1,1,3,3-Tetrachloropropene, 1,3,3,3-Tetrachloropropene, or Combinations Thereof

(a) Process for Preparing 1,1,3,3-tetrachloropropene, 1,3,3,3-tetrachloropropene, or Combinations Thereof.

Another aspect of the present disclosure encompasses a process for preparing 1,1,3,3-tetrachloropropene, 1,3,3,3-tetrachloropropene, or combinations thereof. The process commences by preparing and reacting a mixture comprising 1,1,1,3,3-pentachloropropane (240FA) and an aqueous base in a jet loop reactor in the presence of a phase transfer agent catalyst (PTA). In one embodiment, the PTA comprises a quaternary ammonium salt such as Aliquat 336. The aqueous base is described above in Section (I)(b). In a preferred embodiment, the aqueous base comprises 5 to 10 wt % NaOH, KOH, or combinations thereof and up to saturated wt % of NaCl.

### (b) Reaction Conditions

The reaction conditions are described above in Section (I)(d).

(c) Output from the Process to Prepare 1,1,3,3-tetrachloropropene, 1,3,3,3-tetrachloropropene, or Combinations Thereof.

The 1,1,1,3,3-pentachloropropane fed to the above described process may be converted to 1,1,3,3-tetrachloropropene, 1,3,3,3-tetrachloropropene, or combinations thereof in at least 50% conversion. In various embodiments, the conversion of 1,1,1,3,3-pentachloropropane to 1,1,3,3-tetrachloropropene, 1,3,3,3-tetrachloropropene, or combinations thereof may be at least 50%, at least 60%, at least 75%, at least 85%, at least 95%, and at least 99%.

The selectivity to 1,1,3,3-tetrachloropropene, 1,3,3,3-tetrachloropropene, or combinations thereof can and will vary depending on the reaction conditions, base, the purity level of the 1,1,1,3,3-pentachloropropane, and the 1,1,3,3-tetrachloropropene, 1,3,3,3-tetrachloropropene, or combinations thereof produced. Generally, the selectivity to 1,1,3,3-tetrachloropropene, 1,3,3,3-tetrachloropropene, or combinations thereof may be greater than 70%. In various embodiments, the selectivity to the 1,1,3,3-tetrachloropropene, 1,3,3,3-tetrachloropropene, or combinations thereof may be greater than 70%, greater than 80%, greater than 90%, or greater than 95%. In preferred embodiments, the selectivity to the 1,1,3,3-tetrachloropropene, 1,3,3,3-tetrachloropropene, or combinations thereof may range from 95% to 99%.

(d) Separation of the 1,1,3,3-tetrachloropropene, 1,3,3,3-tetrachloropropene, or Combinations Thereof and Recycling Product Streams.

The process for separating the 1,1,3,3-tetrachloropropene, 1,3,3,3-tetrachloropropene, or combinations thereof from the reactor contents is described above in Section (II). Specific recycle streams useful in improving the efficiency of the process are described above in Section (II).

The first product effluent stream (a) from the separator comprises the 1,1,3,3-tetrachloropropene, 1,3,3,3-tetrachloropropene, or combinations thereof, in a yield of at least about 10%. In various embodiments, the yield is at least about 20%, at least about 50%, at least about 70%, at least about 75%, at least about 80%, at least about 85%, at least about 90%, at least about 95%, or at least about 99%.

### (VI) Further Reaction of the Chlorinated Alkenes

In one aspect, disclosed herein are processes for the conversion of halogenated alkenes, such as 1,1,3-trichloropropene, 3,3,3-trichloropropene, or combinations thereof; 1,1,2,3-tetrachloropropene, 2,3,3,3-tetrachloropropene, or combinations thereof; and 1,1,3,3-tetrachloropropene, 1,3,3,3-tetrachloropropene, or combinations thereof, to one or more hydrofluoroolefins. These processes comprise contacting the halogenated alkenes with a fluorinating agent in the presence of a fluorination catalyst, in a single reaction or two or more reactions. These processes can be conducted in either gas phase or liquid phase with the gas phase being preferred at temperatures ranging from 50° C. to 400° C.

Generally, a wide variety of fluorinating agents can be used. Non-limiting examples of fluorinating agents include HF, F₂, ClF, AlF₃, KF, NaF, SbF₃, SbF₅, SF₄, or combinations thereof. The skilled artisan can readily determine the appropriate fluorination agent and catalyst. Examples of hydrofluoroolefins that may be produced using these processes include, but are not limited to 2,3,3,3-tetrafluoroprop-1-ene (HFO-1234yf), 1,3,3,3-tetrafluoroprop-1-ene (HFO-1234ze), 3,3,3-trifluoroprop-1-ene (HFO-1243zf), and 1-chloro-3,3,3-trifluoroprop-1-ene (HFCO-1233zd).

### Definitions

When introducing elements of the embodiments described herein, the articles “a”, “an”, “the” and “said” should mean there are one or more elements. The terms “comprising”, “including” and “having” are inclusive and mean there may be additional elements other than the listed elements.

The term “113e” refers to 1,1,3-trichloropropene.

The term “333e” refers to 3,3,3-trichloropropene.

The term “1123e” refers to 1,1,2,3-tetrachloropropene.

The term “2333e” refers to 2,3,3,3-tetrachloropropene.

Having described the invention, it will be apparent that modifications and variations are possible without departing from the invention defined in the appended claims.

### EXAMPLES

These examples illustrate various embodiments of the invention.

### Example 1: Exemplary Jet Loop Reactor

FIG. 1 shows the schematic of a 23 cm inner diameter reactor with a two-phase nozzle 2 and a draft tube 3 and an impinging plate 10 below the draft tube 3. The nozzle 2 consists of two tubular sections where the organic liquid mixture 4 enters the tube. The design has a smaller diameter at the outlets of the tube nozzles 2 to provide mixing of the organic 4 and recycle feed 6. The entrance to the two-phase nozzle is placed at the same height as the eductor nozzle entrance to enhance recirculation in the reactor. The impinging plate 10 is placed below the exit of the draft tube 3 so the liquid mixture is forced to be recycled 31, 32 to the top of the reactor. This enhances the mass transfer effects by maintaining the emulsion and hence improving the desired reaction rate.

The reactor of FIG. 1 was used in a batch process to produce a mixture containing 1,1,3- and 3,3,3-trichloropropenes. An equimolar amount of NaOH and 1,1,1,3-tetrachloropropane were combined with 1.8 wt % Aliquat 336. The caustic concentration was 9 wt % in a 16.5 wt % of NaCl/water solution. The reactor was initially filled to cover the nozzle and draft tube with the aqueous and organic reactant such that the caustic is about 0.5 mole % in excess at 18° C. A pump was powered to produce about 650 kg/hr flow rate through the nozzle and about 11 m/s nozzle velocity. After less than a minute the mixture was completely emulsified and after 6 hr the reaction was stopped. The reaction mixture was heated to 62° C. at the end of the reaction to give an average temperature of 39° C. The bottom organic product was analyzed and 91.5% of 1,1,1,3-tetrachloropropane was converted to the trichloropropene isomers product at 93.3% selectivity.

1. A process for preparing at least one chlorinated alkene from at least one chlorinated alkane, the process comprising forming a reaction mixture comprising at least one chlorinated alkane and an aqueous base, and forming a reaction product comprising at least one chlorinated alkene, wherein the process is performed in a jet loop reactor.

2. The process of claim 1, wherein the jet loop reactor comprises a draft tube having a top, a bottom and a diameter, and a nozzle near the top of the draft tube and an impinging plate below the bottom of the draft tube.

3. The process of claim 2 wherein the ratio of the reactor height to the reactor diameter is at least 5 and the ratio of the draft tube height to the reactor diameter is at least 4.

4. The process of claim 2, wherein the ratio of the draft tube inner diameter to the reactor inner diameter is at least 0.6.

5. The process of claim 1, wherein the nozzle has an inner diameter and the ratio of nozzle inner diameter to the diameter of the reactor is at least 0.02.

6. The process of claim 1, wherein the ratio of the impinging plate diameter to inner diameter of the reactor is less than 0.9.

7. A process of claim 1, wherein the at least one chlorinated alkane comprises a C₂-C₆ alkane.

8. The process of claim 1, wherein the at least one chlorinated alkane comprises a chlorinated propane or a chlorinated ethane.

9. The process of claim 1, wherein at least one chlorinated alkane is selected from the group consisting of a dichlorinated propane, a trichlorinated propane, a tetrachlorinated propane, a pentachlorinated propane, a hexachlorinated propane, a tetrachlorinated ethane, trichlorinated ethane, dichlorinated ethane, and combinations thereof.

10. The process of claim 1, wherein the chlorinated alkane comprises 1,1,1,3-tetrachloropropane; 1,1,1,2,3-pentachloropropane; or 1,1,1,3,3-pentachloropropane.

11. The process of claim 1, wherein the at least one chlorinated alkene comprises a chlorinated propene or a chlorinated ethene.

12. The process of claim 1, wherein the chlorinated alkene comprises a monochlorinated propene, a dichlorinated propene, a trichlorinated propene, a tetrachlorinated propene, a pentachlorinated propene, or combinations thereof.

13. The process of claim 12, wherein the chlorinated alkene comprises 1,1,3-trichloropropene, 3,3,3-trichloropropene, or combinations thereof.

14. The process of claim 12, wherein the chlorinated alkene comprises 1,1,2,3-tetrachloropropene; 2,3,3,3-tetrachloropropene, or combinations thereof.

15. The process of claim 12, wherein the chlorinated alkene comprises 1,1,3,3-tetrachloropropene; 1,3,3,3-tetrachloropropene, or combinations thereof.

16. The process of claim 1, wherein the aqueous base is an aqueous solution comprising one or more bases selected from the group consisting of sodium hydroxide, potassium hydroxide, lithium hydroxide, calcium hydroxide, sodium carbonate, potassium carbonate, sodium bicarbonate, potassium bicarbonate, and combinations thereof.

17. The process of claim 16, wherein the base comprises sodium hydroxide.

18. The process of claim 1, wherein the concentration of the aqueous base between about 5 weight percent (wt %) and about 50 wt %.

19. The process of claim 1, wherein the concentration of the aqueous base is between about 5 wt % and about 10 wt %.

20. The process of claim 1, wherein the aqueous base further comprises one or more halide salts comprising NaCl, KCl, CaCl2 or combinations thereof.

21. The process of claim 20, wherein the concentration of the halide salt in the aqueous base is up to saturation.

22. The process of claim 1, wherein the source of aqueous base is one or more cell effluents selected from the group consisting of a diaphragm cell, a membrane cell, and combinations thereof.

23. The process of claim 1, wherein a quaternary ammonium salt phase transfer agent is used as a catalyst.

24. The process of claims 23, wherein the catalyst is Aliquat 336.

25. The process of claim 23, wherein the catalyst concentration is greater than 0.05 wt % and less than 5 wt %.

26. The process of claim 1, wherein the jet loop reactor contains one or more nozzles, one or more draft tubes, optionally one or more feed lines, optionally one or more product outlets, optionally one or more impingement plates, optionally one or more external circuits and optionally further structures.

27. The process of claim 1, wherein the nozzle exit jet velocity is greater than about 1 m/s.

28. The process of claim 27 wherein the jet velocity is greater than 10 m/s.

29. The process of claim 1, wherein the jet loop reactor is operated by a batch, semibatch or continuous process, optionally with recirculation.

30. The process of claim 29, wherein the process is a continuous process.

31. The process of claim 1, wherein the jet loop reactor is built into a cascade of reactors comprising two or more jet loop reactors; or one or more jet loop reactors and one or more jet zone loop reactors; or one or more jet loop reactors and one or more airlift loop reactors; or one or more jet loop reactors and one or more stirred vessels.

32. The process of claim 1, wherein the reaction product is separated from the reaction mixture to form a bottom stream, wherein at least a portion of the bottom stream is recycled to the reactor in a recycle stream.

33. The process of claim 32, wherein fresh feed comprising at least one chlorinated alkane, aqueous base or both is fed to the reactor.

34. The process of claim 33, wherein the recycle stream is fed to the reactor with a recycle stream mass flow, the fresh feed is fed to the reactor with a fresh feed mass flow, and the mass ratio of recycle feed to fresh feed is equal to or greater than 1.

35. The process of claim 1, wherein the temperature of the process is between about 20° C. and about 120° C.

36. The process of claim 1, wherein the pressure of the process between about 0 psig and about 1000 psig.

37. The process of claim 1, wherein the selectivity of the process is at least 70%.

38. The process of claim 1, wherein the conversion of the process is at least 50%.

39. A process of claim 1, the process comprising the caustic cracking of 1,1,1,3-tetrachloropropane with an aqueous base comprising 5-20 wt % NaOH, KOH or a combination thereof and up to saturation salt to form chlorinated alkenes comprising 1,1,3-trichloropropene and 3,3,3-trichloropropene, wherein the process is performed in a jet loop reactor.

40. A process of claim 1, the process comprising the caustic cracking of 1,1,1,2,3-pentachloropropane (240DB) with an aqueous base comprising 5-20 wt % NaOH, KOH or a combination thereof and up to saturated salt to form chlorinated alkenes comprising 1,1,2,3-tetrachloropropene, 2,3,3,3-tetrachloropropene, and combinations thereof, wherein the process is performed in a jet loop reactor.

41. A process of claim 1, the process comprising the caustic cracking of 1,1,1,3,3-pentachloropropane (240FA) with an aqueous base comprising 5-20 wt % NaOH, KOH or combinations thereof and up to saturated salt, to form chlorinated alkenes comprising 1,1,3,3-tetrachloropropene, 1,3,3,3-tetrachloropropene, and combinations thereof, wherein the process is performed in a jet loop reactor.

42. A process of claim 39, wherein the process is performed at a temperature of about 40° C. to about 120° C.

43. A process of claim 39, wherein the chlorinated alkenes are purified.

44. A process of claim 1, wherein the chlorinated alkene is converted into a fluorinated product.

45. The process of claim 2, wherein the nozzle outlet is placed within one diameter of the draft tube above or below the top of the draft tube.

46. The process of claim 45, wherein the nozzle outlet is at the top of the draft tube.

47. The process of claim 45, wherein the flow from the nozzle is directed downward into the top of the draft tube.
